(19) United States
(12) Patent Application Publication
(10) Pub. No.: US 2025/0002901 A1
(43) Pub. Date: Jan. 2, 2025

(54) PROCESSES FOR MEASURING STRAIN FITNESS AND/OR GENOTYPE SELECTION IN BIOREACTORS

(71) Applicant: Inscripta, Inc., Pleasanton, CA (US)

(72) Inventors: Andrew Horwitz, Oakland, CA (US); Amanda Reider Apel, Alameda, CA (US); Theodore M. Tarasow, Moraga, CA (US); Mona Mirsiaghi, Emeryville, CA (US); Christopher Graves, Zionsville, PA (US); Charles Barbieri, Alameda, CA (US)

(21) Appl. No.: 18/704,744

(22) PCT Filed: Oct. 24, 2022
(86) PCT No.: PCT/US2022/047519

§ 371 Date: Apr. 25, 2024

Related U.S. Application Data
(60) Provisional application No. 63/272,126, filed on Oct. 26, 2021.

(51) Int. Cl. C12N 15/10; C12Q 1/06; C40B 30/06

(52) U.S. Cl. CPC C12N 15/1065; C12Q 1/06; C40B 30/06

(57) ABSTRACT

The disclosure provides examples of multi-parallel processes that are used to measure strain fitness of genotypically different strains of a microorganism in one or more bioreactors and the selection of strains that have improved genotypes.

### CROSS REFERENCE TO RELATED APPLICATIONS

This application claims priority under 35 U.S.C. § 119 from Provisional Application Ser. No. 63/272,126 filed Oct. 26, 2021, the disclosure of which is incorporated herein by reference.

### BACKGROUND

Synthetic biology has the potential to produce many new molecules of interest which are challenging to synthesize by traditional chemistry. However, economical bioproduction at industrial scale depends on optimizing many parameters, including growth under bioreactor conditions, achieving high product titers, rates, and yields (TRY), as well as utilization of as many carbon streams derived from renewable carbon feedstocks. While many new molecules can be produced at the laboratory scale, successful development of an economically-viable strain at industrial scale (20,000 L-2,000,000 L) is estimated to cost as much as 1 billion dollars.

### SUMMARY

One goal of synthetic biology is to engineer cells for high performance (productivity, stability) at scale. But the vast majority of strains are assessed under very different conditions in high throughput screening (HTS). Of the 1,000s of strains evaluated in HTS per cycle, only a very few (<1%) can be promoted for further evaluation in screening bioreactors, which model full scale manufacturing conditions.

While prior HTS methodologies can be effective in assessing production of a product of interest, they have proven ineffective in predicted scalability of a given strain of a microorganism. The conditions of HTS do not accurately reflect to complex stresses and conditions of growth in a bioreactor, and therefore relying on HTS as a method for selecting strains that possess a desirable fitness for bioreactor growth has historically proven to be largely unsuccessful. In other words, prior methodologies often do not recognize that growing competing strains of a microorganism in a bioreactor can be a selection in and of itself.

Examples described herein provide multi-parallel processes that are used to measure strain fitness of genotypically different strains of a microorganism in one or more bioreactors and further the selection of strains that have improved genotypes.

Provided herein are screening methods that utilize a tank selection process that allows for the screening of genotypically different strains of a microorganism in one or more bioreactors to measure the frequency at which each genotype is represented throughout the bioreactor run. The data set generated therefrom can be analyzed using software and algorithms to generate quantitative fitness scores for each strain genotype. In parallel, these same genotypically different strains can be assessed by standard HTS methods, e.g., product titer and biomass yield. The fitness scores from the tank selection process can then be overlaid upon the data generated using the HTS process in a multivariate tank promotion model. By gaining a detailed understanding of tank fitness at this stage, it is possible to make much better promotion decisions of strains for standard bioreactor screening.

In a particular implementation, the disclosure provides a tank selection process for correlating a genotype from a strain of a microorganism with its fitness in a bioreactor(s), comprising: (i) seeding one or more bioreactors with a plurality of genotypically different strains of a microorganism, wherein each strain of the microorganism comprises a unique identifying tag; (ii) culturing the genotypically different strains in one or more bioreactors, wherein, at various time points, samples are taken from the one or more bioreactors; (iii) measuring the frequency of each genotypically different strain in the samples, wherein each genotypically different strain can be identified by the unique identifying tag; and (iv) assigning a quantitative fitness score for each of the genotypically different strains of the microorganism based upon how frequently the particular strain is present in the samples, wherein the value of the quantitative fitness score is correlative with the genotype from a strain and its fitness in bioreactor(s). In a further implementation, the microorganism is a bacterium or a yeast. In yet a further implementation, the yeast is selected from the group consisting of Saccharomyces cerevisiae, Pichia pastoris, Hansenula polymorpha, Kluyveromyces lactis, Schizosaccharomyces pombe, Yarrowia lipolytica, and Arxula adeninivorans. In another implementation, the yeast is Saccharomyces cerevisiae. In yet another implementation, the bacterium is selected from the group consisting of Escherichia Coli, Caulobacter crescentus, Rodhobacter sphaeroides, Pseudoalteromonas haloplanktis, Shewanella sp. strain Ac10, Pseudomonas fluorescens, Pseudomonas putida, Pseudomonas aeruginosa, Halomonas elongate, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, and Lactobacillus gasseri. In a certain implementation, the bacterium is Escherichia Coli. In a further implementation, the microorganism is a recombinantly modified microorganism. In yet a further implementation, the microorganism is recombinantly modified to express a biofuel, a terpene or terpene containing compound, an alkaloid, a phenylpropanoid, a fatty acid-derived compound, a polyketide, or a recombinant protein. In another implementation, the genotypically different strains of the microorganism are generated using mutagenesis or gene editing. In yet another implementation, the genotypically different strains of the microorganism comprise a mutation, edit, or disruption in a single gene of the microorganism. In a further implementation, the genotypically different strains of the microorganism comprise 2 to 10 mutations, edits, or disruptions in one or more genes of the microorganism. In yet a further implementation, the genotypically different strains of the microorganism are generated by using a gene editing technology selected from the group consisting of CRISPR-Cas12a, CRISPR-Cas13, CRISPR-Cas9, TALEN, and Zinc Finger Nucleases. In a certain implementation, the genotypically different strains of the microorganism are generated by using CRISPR-Cas12a. In another implementation, the genotypically different strains of the microorganism are generated by using a digital genome engineering platform. In yet another implementation, the unique identifying tag is a barcode sequence. In a certain implementation, the barcode sequence is incorporated into the genome of the different strains of the microorganism using CRISPR-Cas12a. In another implementation, the CRISPR-Cas12a plasmids used to edit the strain's genome further comprise the barcode sequences.

In a particular implementation, the disclosure also provides a screening process to identify strain(s) of a microorganism that exhibit superior strain fitness in bioreactors and improved genotypes, the screening process comprising a tank selection process and a high throughput screening process: the tank selection process comprising: (i) seeding one or more bioreactors with a plurality of genotypically different strains of a microorganism, wherein each strain of the microorganism comprises a unique identifying tag; (ii) culturing the genotypically different strains in one or more bioreactors, wherein, at various time points, samples are taken from the one or more bioreactors; (iii) measuring the frequency of each genotypically different strain in the samples, wherein each genotypically different strain can be identified by the unique identifying tag; (iv) assigning a quantitative fitness score for the genotypically different strains of the microorganism based upon how frequently the particular strain is present in the samples, wherein superior strain fitness in bioreactors is correlative with a high quantitative fitness score; and the high throughput screening process comprising: (a) generating isolates from the plurality of genotypically different strains of the microorganism, wherein each isolate comprises only one strain of the microorganism, wherein each isolate can be identified by the unique identifying tag; (b) assessing the isolates for improved genotypes in comparison to the microorganism or other isolates by using high throughput screening, wherein strain(s) of the microorganism that exhibit superior strain fitness in bioreactors and improved genotypes can be identified by having a high quantitative fitness score and exhibiting an improved genotype in high throughput screening. In a further implementation, the microorganism is a bacterium or a yeast. In yet a further implementation, the yeast is selected from the group consisting of Saccharomyces cerevisiae, Pichia pastoris, Hansenula polymorpha, Kluyveromyces lactis, Schizosaccharomyces pombe, Yarrowia lipolytica, and Arxula adeninivorans. In another implementation, the yeast is Saccharomyces cerevisiae. In yet another implementation, the bacterium is selected from the group consisting of Escherichia Coli, Caulobacter crescentus, Rodhobacter sphaeroides, Pseudoalteromonas haloplanktis, Shewanella sp. strain Ac10, Pseudomonas fluorescens, Pseudomonas putida, Pseudomonas aeruginosa, Halomonas elongate, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, and Lactobacillus gasseri. In a further implementation, the bacterium is Escherichia Coli. In yet a further implementation, the microorganism is a recombinantly modified microorganism. In another implementation, the microorganism is recombinantly modified to express a biofuel, a terpene or terpene containing compound, an alkaloid, a phenylpropanoid, a fatty acid-derived compound, a polyketide, or a recombinant protein. In yet another implementation, the genotypically different strains of the microorganism are generated using mutagenesis or gene editing. In a further implementation, the genotypically different strains of the microorganism comprise a mutation, edit, or disruption in a single gene of the microorganism.

In yet a further implementation, the genotypically different strains of the microorganism comprise 2 to 10 mutations, edits, or disruptions in one or more genes of the microorganism. In another implementation, the genotypically different strains of the microorganism are generated by using a gene editing technology selected from the group consisting of CRISPR-Cas12a, CRISPR-Cas13, CRISPR-Cas9, TALEN, and Zinc Finger Nucleases. In yet another implementation, the genotypically different strains of the microorganism are generated by using CRISPR-Cas12a. In a further implementation, the genotypically different strains of the microorganism are generated by using a digital genome engineering platform. In yet a further implementation, the unique identifying tag is a barcode sequence. In a certain implementation, the barcode sequence is incorporated into the genome of the different strains of the microorganism using CRISPR-Cas12a. In another implementation, the CRISPR-Cas12a plasmids used to edit the strain's genome further comprise the barcode sequences. In yet another implementation, the high throughput screening process is run in parallel with the tank selection process. In a further implementation, the high throughput screening process is used to determine and isolate strains which have an improved genotype for product formation. In yet a further implementation, the product that is formed is selected from the group consisting of a biofuel, a terpene or terpene containing compound, an alkaloid, a phenylpropanoid, a fatty acid-derived compound, a polyketide, and a recombinant protein. In another implementation, the biofuel is an alcohol selected from the group consisting of methanol, ethanol, propanol, butanol, and isobutanol. In yet another implementation, the terpene or terpene containing compound is selected from the group consisting of β-Carotene, camphor, menthol, limonene, linalool, geraniol, farnesene, squalene, capsidiol, and artemisinin. In a further implementation, the terpene or terpene containing compound is farnesene. In another implementation, the fatty acid-derived compound is selected from the group consisting of free fatty acids, fatty alcohols, fatty acid ethyl esters, and fatty acid methyl esters. In yet another implementation, the recombinant protein is selected from the group consisting of an antibiotic, a restriction enzyme, a hormone, a protein therapeutic, a xylanase, a protease, a vaccine, a polyphenol oxidase, a laccase, and a cystatin.

In another aspect, the present disclosure provides a process, comprising: seeding a bioreactor with a library of genetically modified strains of a microorganism, wherein each strain in the library comprises a unique tag and a unique genetic modification; culturing the library of genetically modified strains in the bioreactor; and determining fitness of a strain, which is part of the library, from a sample from the bioreactor, using at least an amount of that strain present in the sample.

In some implementations, determining fitness of the strain in the library comprises quantifying the amount of the strain that is present in the sample.

In some implementations, the microorganism is a bacterium or a yeast.

In some implementations, the microorganism is a yeast selected from the group consisting of Saccharomyces cerevisiae, Pichia pastoris, Hansenula polymorpha, Kluyveromyces lactis, Schizosaccharomyces pombe, Yarrowia lipolytica, and Arxula adeninivorans. In some implementations, the yeast is Saccharomyces cerevisiae.

In some implementations, the microorganism is a bacterium selected from the group consisting of Escherichia coli, Caulobacter crescentus, Rodhobacter sphaeroides, Pseudoalteromonas haloplanktis, Shewanella sp. strain Ac10, Pseudomonas fluorescens, Pseudomonas putida, Pseudomonas aeruginosa, Halomonas elongate, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, and Lactobacillus gasseri. In some implementations, the bacterium is Escherichia coli.

In some implementations, the unique genetic modification comprises an insertion, deletion, or substitution of one or more nucleic acids.

In some implementations, the unique genetic modification comprises a deletion of an endogenous gene, downregulation of an endogenous gene, or upregulation of an endogenous gene.

In some implementations, the unique genetic modification is made using a gene editing technology selected from the group consisting of CRISPR-Cas12a, CRISPR-Cas13, CRISPR-Cas9, TALEN, and Zinc Finger Nucleases.

In some implementations, each strain in the library is recombinantly modified to express a heterologous gene.

In some implementations, each strain in the library is recombinantly modified to produce or increase production of a biofuel, a terpene or terpene containing compound, an alkaloid, a phenylpropanoid, a fatty acid-derived compound, a polyketide, or a recombinant protein.

In some implementations, each strain is recombinantly modified using a gene editing technology selected from the group consisting of CRISPR-Cas12a, CRISPR-Cas13, CRISPR-Cas9, TALEN, and Zinc Finger Nucleases.

In some implementations, the unique tag is a barcode sequence. In some implementations, the process can further comprise incorporating the barcode sequence into the microorganisms' genome using a gene editing technology selected from the group consisting of CRISPR-Cas12a, CRISPR-Cas13, CRISPR-Cas9, TALEN, and Zinc Finger Nucleases.

In some implementations, the fitness is determined for each strain in the library.

In some implementations, determining fitness comprises assigning a quantitative fitness score to at least one strain in the library.

In some implementations, a sample is removed from the bioreactor at two or more time points.

In some implementations, the process may further comprise assessing one or more strains in the library by high throughput screening. In some implementations, the high throughput screening comprises determining production titer of a product. In some implementations, the product is selected from the group consisting of a biofuel, a terpene or terpene containing compound, an alkaloid, a phenylpropanoid, a fatty acid-derived compound, a polyketide, and a recombinant protein. In some implementations, the biofuel is an alcohol selected from the group consisting of methanol, ethanol, propanol, butanol, and isobutanol. In some implementations, the terpene or terpene containing compound is selected from the group consisting of β-Carotene, camphor, menthol, limonene, linalool, geraniol, farnesene, squalene, capsidiol, and artemisinin. In some implementations, the fatty acid-derived compound is selected from the group consisting of free fatty acids, fatty alcohols, fatty acid ethyl esters, and fatty acid methyl esters. In some implementations, the recombinant protein is selected from the group consisting of an antibiotic, a restriction enzyme, a hormone, a protein therapeutic, a xylanase, a protease, a vaccine, a polyphenol oxidase, a laccase, and a cystatin.

In another aspect, the present disclosure provides a process, comprising: culturing a library of genetically modified strains of a microorganism in a bioreactor, wherein each strain in the library comprises a unique tag and a unique genetic modification; and determining fitness for bioreactor growth of a strain in the library by measuring how frequently the strain is present in a sample taken from the bioreactor.

In some implementations, the process may further comprise high throughput screening of the strain or the library. In some implementations, the high throughput screening comprises determining production titer of a product.

In some implementations, the microorganism is a bacterium or a yeast.

In some implementations, the unique genetic modification comprises an insertion, deletion, or substitution of one or more nucleic acids.

In some implementations, the unique genetic modification comprises a deletion of an endogenous gene, downregulation of an endogenous gene, or upregulation of an endogenous gene.

In some implementations, the unique genetic modification is made using a gene editing technology selected from the group consisting of CRISPR-Cas12a, CRISPR-Cas13, CRISPR-Cas9, TALEN, and Zinc Finger Nucleases.

In some implementations, each strain in the library is recombinantly modified to express a heterologous gene.

In some implementations, each strain in the library is recombinantly modified to produce or increase production of a biofuel, a terpene or terpene containing compound, an alkaloid, a phenylpropanoid, a fatty acid-derived compound, a polyketide, or a recombinant protein.

In some implementations, each strain is recombinantly modified using a gene editing technology selected from the group consisting of CRISPR-Cas12a, CRISPR-Cas13, CRISPR-Cas9, TALEN, and Zinc Finger Nucleases.

In some implementations, the unique tag is a barcode sequence. In some implementations, the process may further comprise incorporating the barcode sequence into the microorganisms' genome using a gene editing technology selected from the group consisting of CRISPR-Cas12a, CRISPR-Cas13, CRISPR-Cas9, TALEN, and Zinc Finger Nucleases.

In some implementations, the fitness for bioreactor growth is determined for each strain in the library.

In some implementations, the determining fitness for bioreactor growth comprises assigning a quantitative fitness score to at least one strain in the library.

In some implementations, a sample is removed from the bioreactor at two or more time points during culturing.

It should be appreciated that all combinations of the foregoing concepts and additional concepts discussed in greater detail below (provided such concepts are not mutually inconsistent) are contemplated as being part of the inventive subject matter disclosed herein and may be employed in any combination to achieve the benefits described herein.

### DESCRIPTION OF DRAWINGS

FIG. 1 provides an implementation of a tank selection process disclosed herein using libraries generated by the Inscripta Onyx. Any barcoded library format, however, may be used. Barcode lineage tracking is used to derive fitness scores for 1,000s of genotypes that can be used to improve tank correlation models and promotion decisions. Not shown-singulation and selection of colonies from various sampling timepoints for direct testing of fit populations in HTS.

FIG. 2 provides, in one example, results of a tank selection process disclosed herein with singulated colonies from a final tank selection timepoint assessed in HTS for farnesene production. Ladder_CENPK is wild type yeast, Ladder_Blank is medium only, Ladder_STR063 is the parent strain from strain bank, Bay 170_Sample11_130 is STR063 from bioreactor, and the diagonally shaded bars are isolates from the tank selection. Error bars are standard deviation.

### DETAILED DESCRIPTION

As used herein and in the appended claims, the singular forms “a,” “an,” and “the” include plural referents unless the context clearly dictates otherwise. Thus, for example, reference to “a yeast strain” includes a plurality of such yeast strains and reference to “the barcode” includes reference to one or more barcodes and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various implementations use the term “comprising,” those skilled in the art would understand that in some specific instances, an implementation can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some implementations of the present invention, the term “substantially” is defined as approaching a specific numeric value or target to within 20%, 15%, 10%, 5%, or within 1% of the value or target as well as the recited value or target. In further implementations of the present invention, the term “substantially” is defined as approaching a specific numeric value or target to within 1%, 0.9%, 0.8%, 0.7%, 0.6%, 0.5%, 0.4%, 0.3%, 0.2%, or 0.1% of the value or target or the recited value or target.

As used herein, the term “about” is used to indicate that exact values are not necessarily attainable. Therefore, the term “about” is used to indicate this uncertainty limit. In some implementations of the present invention, the term “about” is used to indicate an uncertainty limit of less than or equal to ±20%, ±15%, ±10%, ±5%, or ±1% of a specific numeric value or target as well as the recited numeric value or target. Thus, by way of example, “about 10” should be understood to mean “10” and a range no larger than “8-12”. In some implementations of the present invention, the term “about” is used to indicate an uncertainty limit of less than or equal to: ±1%, ±0.9%, ±0.8%, ±0.7%, 0 6%, ±0.5%, ±0.4%, ±0.3%, ±0.2%, or ±0.1% of a specific numeric value or target as well as the recited numeric value or target.

A “vector” or “recombinant vector” is a nucleic acid molecule that is used as a tool for manipulating a nucleic acid sequence of choice or for introducing such a nucleic acid sequence into a microorganism. A vector may be suitable for use in cloning, sequencing, or otherwise manipulating one or more nucleic acid sequences of choice, such as by expressing or delivering the nucleic acid sequence(s) of choice into a microorganism to form a recombinant microorganism. Such a vector typically contains heterologous nucleic acid sequences not naturally found adjacent to a nucleic acid sequence of choice, although the vector can also contain regulatory nucleic acid sequences (e.g., promoters, untranslated regions) that are naturally found adjacent to the nucleic acid sequences of choice or that are useful for expression of the nucleic acid molecules.

A vector can be either RNA or DNA, either prokaryotic or eukaryotic, and typically is a plasmid. The vector can be maintained as an extrachromosomal element (e.g., a plasmid) or it can be integrated into the chromosome of a recombinant host cell. The entire vector can remain in place within a host cell, or under certain conditions, the plasmid DNA can be deleted, leaving behind the nucleic acid molecule of choice. An integrated nucleic acid molecule can be under chromosomal promoter control, under native or plasmid promoter control, or under a combination of several promoter controls. Single or multiple copies of the nucleic acid molecule can be integrated into the chromosome. A recombinant vector can contain at least one selectable marker.

The term “expression vector” refers to a recombinant vector that is capable of directing the expression of a nucleic acid sequence that has been cloned into it after insertion into a microorganism. A nucleic acid sequence is “expressed” when it is transcribed to yield an mRNA sequence. In most cases, this transcript will be translated to yield an amino acid sequence. The cloned gene is usually placed under the control of (i.e., operably linked to) an expression control sequence. The phrase “operatively linked” refers to linking a nucleic acid molecule to an expression control sequence in a manner such that the molecule can be expressed when introduced (i.e., transformed, transduced, transfected, conjugated and/or conduced) into a microorganism.

Vectors and expression vectors may contain one or more regulatory sequences or expression control sequences. Regulatory sequences broadly encompass expression control sequences (e.g., transcription control sequences or translation control sequences), as well as sequences that allow for vector replication in a host cell. Transcription control sequences are sequences that control the initiation, elongation, and/or termination of transcription. Suitable regulatory sequences include any sequence that can function in a microorganism into which the recombinant nucleic acid molecule is to be introduced, including those that control transcription initiation, such as promoter, enhancer, terminator, operator, and/or repressor sequences. Additional regulatory sequences include translation regulatory sequences, origins of replication, and other regulatory sequences that are compatible with the recombinant cell. The expression vectors may contain elements that allow for constitutive expression or inducible expression of the protein or proteins of interest. Numerous inducible and constitutive expression systems are known in the art.

In some implementations, an expression vector includes at least one nucleic acid molecule of interest operatively linked to one or more expression control sequences (e.g., transcription control sequences or translation control sequences). In one implementation, an expression vector may comprise a nucleic acid encoding a recombinant polypeptide, as described herein, operably linked to at least one regulatory sequence. It should be understood that the design of the expression vector may depend on such factors as the choice of the host cell to be transformed and/or the type of polypeptide to be expressed.

Expression and recombinant vectors may contain a selectable marker, a gene encoding a protein necessary for survival or growth of a host cell transformed with the vector The presence of this gene allows growth of only those host cells that express the vector when grown in the appropriate selective medium Typical selection genes encode proteins that confer resistance to antibiotics or other toxic substances, complement auxotrophic deficiencies, or supply critical nutrients not available from a particular medium. Markers may be an inducible or non-inducible gene and will generally allow for positive selection. Non-limiting examples of selectable markers include the ampicillin resistance marker (i.e., beta-lactamase), tetracycline resistance marker, neomycin/kanamycin resistance marker (i.e., neomycin phosphotransferase), dihydrofolate reductase, glutamine synthetase, and the like. The choice of the proper selectable marker will depend on the microorganisms, and appropriate markers for different microorganisms as understood by those of skill in the art.

Any suitable expression vectors that include (or may be derived from) plasmid vectors, such as those commonly available from commercial sources, may be employed. Vectors can contain one or more replication and inheritance systems for cloning or expression, one or more markers for selection in the host, and one or more expression cassettes. The inserted coding sequences can be synthesized by standard methods, isolated from natural sources, or prepared as hybrids. Ligation of the coding sequences to transcriptional regulatory elements or to other amino acid encoding sequences can be carried out using established methods. A large number of vectors, including bacterial, yeast, and mammalian vectors, have been described for replication and/or expression in various host cells or cell-free systems, and may be used with the sequences described herein for simple cloning or protein expression.

A nucleic acid molecule or polynucleotide can include a naturally occurring nucleic acid molecule that has been isolated from its natural source or produced using recombinant DNA technology (e.g., polymerase chain reaction (PCR) amplification, cloning) or chemical synthesis. Isolated nucleic acid molecules can include, for example, genes, natural allelic variants of genes, coding regions or portions thereof, and coding and/or regulatory regions modified by nucleotide insertions, deletions, substitutions, and/or inversions in a manner such that the modifications do not substantially interfere with the nucleic acid molecule's ability to encode a polypeptide or to form stable hybrids under stringent conditions with natural gene isolates. An isolated nucleic acid molecule can include degeneracies. As used herein, nucleotide degeneracy refers to the phenomenon that one amino acid can be encoded by different nucleotide codons. Thus, the nucleic acid sequence of a nucleic acid molecule that encodes a protein or polypeptide can vary due to degeneracies.

All publications and patents and patent applications mentioned in this disclosure are explicitly incorporated herein by reference in their entirety for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular implementations only and is not intended to limit the scope of the present invention, which is defined solely by the claims.

One goal of synthetic biology is to engineer cells for high performance (productivity, stability) at scale. In some implementations, the vast majority of strains are assessed under very different conditions in high throughput screening (HTS). Of the 1,000s of strains evaluated in HTS per cycle, only a very few (<1%) can be promoted for further evaluation in screening bioreactors, which model full scale manufacturing conditions. A key parameter for successful scale up is strain fitness, which can be approximated by growth rate under bioreactor conditions. Strain fitness is directly related to stability of production-unfit strains are rapidly outcompeted by naturally arising mutants that have acquired a fitness advantage by reducing their productivity. However, assessing growth rates is difficult and expensive even at HTS scale, as it is a kinetic assay involving sampling over multiple timepoints. Currently it may be difficult to assess the fitness of large numbers of strains under relevant bioreactor conditions. This means that, in some instances, an important indicator of strain success at scale cannot be considered at high throughput during the strain development and selection phase, and this introduces considerable risk to the scaleup process.

Historically, strain improvement approaches have been dominated by random mutagenesis or directed evolution, followed by high-throughput screening to identify novel or enhanced phenotypes in existing microbial hosts. While this approach may not introduce novel enzymatic functionalities, it allows for strain improvement without an in-depth understanding of microbial metabolism and potentially searches the entire host genome for beneficial mutations. Candidate strains that undergo random mutagenesis are first screened using a high-throughput method (e.g., 96-well plates) before being validated in a (lower throughput) bioreactor experiment that can more accurately reproduce industrial production conditions. The development of recombinant DNA technology in the 1980s paved the way for targeted genetic modifications of microbial production hosts, including gene knock-outs, knock-downs or overexpression of endogenous genes, as well as the introduction of heterologous genes with new enzymatic functionalities. However, early genetic engineering methods may be labor intensive and low-throughput. As a result, genetic edits are rationally selected, with a focus on well-characterized genes and pathways likely to have positive effects on strain performance. Each individual strain may be tested directly in a bioreactor under conditions specifically optimized for its performance. This strategy led to impressive progress for several products, including 1,3-propanediol, insulin, and amino acid production. However, rational strain engineering efforts are hampered by the contemporary, limited understanding of the microbial metabolism, and low success rates are likely for unstudied pathways and organisms. The emergence of “-omics” technologies (e.g., transcriptomics, proteomics, metabolomics, fluxomics) in the 1990s and the corresponding advances in metabolic modeling significantly increased the success rate of rational strain design. However, despite these technological advances, the complexity of the microbial metabolism in some implementations may be an obstacle during rational strain improvement attempts. As a result, the introduction of (rational) genetics edits derived from these large datasets and metabolic models does not always improve strain performance to the desired level.

Recent advances in DNA synthesis, DNA sequencing, genetic engineering, and automated strain engineering have enabled empirical strategies that address the limitations of rational strain engineering given the complexity of cellular metabolism. Rather than attempting to first understand the metabolic ‘problem’ before designing a small set of targeted genetic ‘solutions’, high-throughput strain construction and testing strategies enable an empirical approach that starts with the ‘solutions’ first. Once thousands of strains are designed, built, and tested, analyzing which of these genetic modifications affect performance can identify the areas of metabolism that are relevant to product formation. This circumvents the need to capture the complexity of microbial metabolism in a single comprehensive model.

Provided herein in one implementation is a tank selection process that enables the screening of a large number of strains under relevant bioreactor conditions. A bioreactor can be inoculated with a seed vial bearing a clonal population of cells with the goal of understanding how a single strain performs. In one implementation of tank selection, a library of barcoded strains bearing diverse genome edits may be used to inoculate the bioreactor. These diverse strains compete for resources in the tank. Samples are taken throughout the process (seed train through main bioreactor). Barcode lineage tracking is performed on extracted DNA samples to measure the frequency at which each genotype is represented throughout the bioreactor run. This generates a massive data set that can be computer analyzed using algorithms to generate quantitative fitness scores for each genotype in the library. In parallel, these same libraries may be assessed by standard HTS methods, e.g., product titer and biomass yield. The fitness scores from the tank selection can then be overlaid upon the HTS data in a multivariate tank promotion model. By gaining a detailed understanding of tank fitness at this stage, it is possible to make much better promotion decisions of strains for standard bioreactor screening.

In addition to deriving an invaluable data set, samples of live cells from each timepoint can be further singulated to colonies and assessed in HTS and genotyped individually to understand how major populations perform. In one implementation, by the final timepoints of a tank selection, certain genotypes dominate the population owing to superior fitness. This pre-selection of the library biases the distribution of strains towards those with high bioreactor fitness. This parameter can be tuned by sampling earlier or later timepoints. By focusing the HTS effort on these populations, the tank correlation is dramatically improved.

By definition, a bioreactor is a vessel in which a biological reaction or change takes place. The biological systems involved include enzymes, microorganisms, animal cells, plant cells, and tissues. In one implementation, the bioreactor is a place where an optimum external environment is provided to meet the needs of the biological reaction system so that a high yield of the bioprocess is achieved. In one implementation, there are complicated interactions between the biological system and the physical and chemical aspects of this process. To design an appropriate bioreactor for a particular bioprocess, intensive studies on the biological system, such as cell growth and metabolism, genetic manipulation, and protein or other product expression are needed to understand the cells' requirement on their physical and chemical environment. A variety of bioreactor types and configurations have thus been exploited and developed along with the advances in the understanding of biological systems. In addition, control the bioreactor's operating parameters can favor the desired functions of the living cells or enzymes. Dissolved oxygen concentration, pH, temperature, mixing, and supplementation of nutrients all need to be controlled and optimized.

Because of the rapid advances in recombinant DNA technology and genome sequencing, the same product or biological process may be achieved by different biological systems: microorganisms, plant cells, animal cells, or enzymes. Their genetic expressions, metabolic manipulation, and bioreaction pathways may be examined. In an attempt to identify the parameters related to the medium for the efficient performance of the chosen biological system, in one implementation the medium design and optimization can be based on a basic knowledge of stoichiometry and experimental data, including monitoring the composition changes of the medium, intermediates, products, and nutrients. Stoichiometric calculations provide quantitative relationships between yields of biomass and product synthesis, maintenance criteria and energy production. Complementing stoichiometric data for the design of a bioreactor, a kinetics study will reveal the biological reaction rates, including cell growth, substrate consumption, product synthesis and by-product formation rates. Many enzymatic reactions may be involved, and inhibitions caused by products, byproducts, or even substrate at high concentrations may be often observed. On the other hand, the physical environment directly affects the biological performance. Shear stress, mass transfer, mixing, pH and temperature may be all interrelated and can influence biological reactions. Also, other important factors to be considered are downstream process parameters. With this understanding of the biological system and its parameters on its physical and chemical environment, a proper bioreactor type can be selected. Among the bioreactor types available for a certain bioprocess, it is important to have a balanced consideration of many factors, including oxygen transfer, mixing, shear, operational stability and reliability, scale-up, and cost. The chosen bioreactor should be further characterized and the operational mode should be optimized. The bioreactor's characteristics and operational mode also may affect the biological performances. An efficient bioreactor system can rely on its control and support systems. Other process parameters and constraints may also be considered when integrating a bioreactor system into a larger production system.

The cell culture in the bioreactor may be mixed thoroughly after different periods. This mixing of cultures is done with the help of specially designed impellers of the bioreactor. Mixing can be used to obtain a homogenous division of nutrients and temperature, as well as for adjusting the pH around the cell culture. All this is done to avoid unequal distribution of pH, temperature, and nutrients. It should be strictly kept in mind that the mixing should be handled with care as vigorous mixing can actually destroy the cells and may result in genetic modification.

A biological active organism or environment may be sensitive to temperature, often working well under a specified range of temperatures. Any enzyme can lower its function when exposed to a very high temperature or very low temperature. Therefore, the temperature value deemed suitable for the cell culture to proliferate in the bioreactor may be maintained and adjusted as needed. In one implementation, the desired temperature values for mammalian enzymes are about 37° C. In one implementation, mammalian cells, insect cells, and plant cells may all share the same range of ideal temperature—from 25 to 37° C. In one implementation, the ideal temperature for bacteria, yeast, and fungi cells ranges from 20 to 60° C.

A bioreactor's temperature should be kept constant throughout the entire procedure. However, the temperature sometimes may be lowered at the very end of the reaction process in order to obtain valuable byproducts to be used later on. These byproducts may include penicillin and other recombinant proteins. This function is known as the temperature shift—in one implementation, this technique is used to store the finished product in its stable condition.

The maintenance of pH value may be an important factor within the bioreactor process. Having an unbalanced pH can have negative impacts such as altering the desired results or even damaging the cell culture. This is why that, in one implementation, bioreactors have pH sensors that are used to measure the pH value at all times. Buffers are also commonly present within the media system, which can help avoid any sudden changes in pH.

In one implementation, the nutrients consumed by the cell culture are basic and organic. For example, the main nutrient components may include water, glucose, carbon, nitrogen, phosphorous, salts, minerals, and trace elements. The various vitamins and essential amino acids included may be different depending on the organisms being grown in the bioreactor.

In one implementation, a mixture of gases is introduced in the bioreactor for the growth of the cell culture. In one implementation, the constant stirring may equally distribute the gas (oxygen) to the culture medium. In one implementation, the higher the pressure in the vessel, the more the oxygen is dissolved. In one implementation, due to these high pressures, bioreactors are designed with stainless steel rather than glass or other materials. If there is an anaerobic medium, then gas may become optional.

In one implementation, the prevention of foam is highly recommended in bioreactors because it blocks the way of the flow of gases. Either antifoam control systems or antifoam agents may be used. These chemicals are used differently according to the cell culture because these antifoam chemicals can react with the enzymes.

With respect to its mode of operation, the bioreactor process may be divided into: batch, fed-batch, perfusion, and continuous processing. A batch process may be a relatively simple bioreactor process. In one implementation, a bioreactor is filled with a certain amount of medium, then the cells are inoculated into the bioreactor. The cell culture will consume nutrients and grow, and then the run is harvested. A finite and defined quantity of product is obtained at the end. This process may be relatively easy to control and maintain, as small effort is needed. The batch process is often used in pharmaceuticals and specialty chemicals.

The fed-batch process is similar to the batch process, except one extra step is added. Instead of filling a bioreactor just once at the beginning with a medium and inoculated cells, a fed-batch process involves adding in fresh ‘feeds’ of nutrients throughout the run. This may allow additional cell growth and may result in a higher product yield.

As the name suggests, continuous processing is a continuous process. In one implementation, a bioreactor reaches a steady state, where the growth rate of cells is equal to the death rate. In one implementation, a continuous stream of feed may be added to the bioreactor, and a continuous feed of product may be coming out of the bioreactor. This may allow the process to have the potential to run indefinitely, with no downtime between batches, no batch variability, and no expensive cleaning tasks involved. However, in many instances continuous processing is still currently in relatively early stages of development and may involve a sophisticated operating and controlling system.

One common bioreactor for industrial applications is the stirred tank reactor (STR), due to its high flexibility and low operating costs. Four main types of stirred tank bioreactors by application include: microbial bioreactors/fermenters (bacteria, yeast, fungi); cell culture bioreactors (mammalian cells, insect cells); single-use or disposable bioreactors (SUB's used for cell culture); and bioreactors for special applications (SSF and Photobioreactors)

A microbial stirred tank reactor can be equipped for rapid, high-shear mixing and good oxygen transfer. A tall height/diameter aspect ratio may be used to let bubbles of gas remain in contact with the culture for as long as possible. Multiple, flat-bladed Rushton turbine impellors break up gas bubbles with high shear forces to increase the surface area for gas exchange. In one implementation, gas transfer rates are usually high, and foaming may become an issue. Microbial cultures have a long history in food production, industrial applications and the pharmaceuticals (white biotechnology).

In a particular implementation, the disclosure provides a tank selection process for correlating the genotypes of strain(s) of a microorganism with its fitness in a bioreactor(s), comprising the step of seeding one or more bioreactors with a plurality of genotypically different strains of a microorganism, wherein each strain of the microorganism comprises a unique identifying tag. In a further implementation, the microorganism is a bacterium, a fungus, an alga, or an archaeon. The bacterium may be of any species, for example one selected from the group consisting of Escherichia Coli, Caulobacter crescentus, Rodhobacter sphaeroides, Pseudoalteromonas haloplanktis, Shewanella sp. strain Ac10, Pseudomonas fluorescens, Pseudomonas putida, Pseudomonas aeruginosa, Halomonas elongate, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, and Lactobacillus gasseri. In a particular implementation, the microorganism is Escherichia Coli. The fungus may be of any species, for example, one selected from the group consisting of Saccharomyces cerevisiae, Pichia pastoris, Hansenula polymorpha, Kluyveromyces lactis, Schizosaccharomyces pombe, Yarrowia lipolytica and Arxula adeninivorans. In a certain implementation, the microorganism is Saccharomyces cerevisiae. The microorganism may be a wild type microorganism or a recombinantly modified microorganism. For example, the microorganism may be recombinantly modified to express heterologous genes, to downregulate certain endogenous enzymes or proteins, and/or to upregulate certain endogenous enzymes or proteins. In a certain implementation, the microorganism may be recombinantly modified to produce or increase production of a biofuel (e.g., methanol, ethanol, propanol, butanol, or isobutanol). Alternatively, the microorganism may be recombinantly modified to produce any number of commercially valuable products, including but not limited to: terpene or terpene containing compounds (e.g., β-Carotene, camphor, menthol, limonene, linalool, geraniol, farnesene, squalene, capsidiol, artemisinin, etc.); fatty acid-derived compounds (e.g., free fatty acids, fatty alcohols, fatty acid ethyl esters, fatty acid methyl esters, etc.); or recombinant proteins (e.g., antibiotics, restriction enzymes, hormones, therapeutics, xylanases, proteases, vaccines, polyphenol oxidases, laccases, cystatins, etc.).

The plurality of genotypically different strains of a microorganism can be generated using various methods, including by mutagenesis or by gene editing. In regards to mutagenesis, transposon systems (e.g., a Sleeping Beauty transposon system) have been developed that provide for the generation of libraries of genotypically different strains of a microorganism. These transposon systems also allow for tagging individual strains with a unique identifying tag (i.e., barcode sequences) (e.g., see Santiago et al., BMC Genomics 16 (252) (2015)). In regards to gene editing, various gene editing tools may be used to generate libraries of genotypically different strains of a microorganism, including CRISPR-Cas based systems (e.g., CRISPR-Cas12a, CRISPR-Cas9, CRISPR-Cas13, etc.), TALEN, and zinc finger nucleases. One example of the gene editing tools includes CRISPR-Cas9 (see Ran et al., Nature Protocols 8:2281-2308 (2013)). Various commercial systems have been developed using CRISPR-Cas based systems to make libraries of genotypically different strains of a microorganism, including the Onyx™ digital genome engineering system by Inscripta Inc. (USA). The gene editing tools can also allow for tagging individual strains with a unique identifying tag (i.e., barcode sequences). Further, the above mutagenesis and gene editing systems can be used to study the effects of more than 1 mutation or disruption in a single gene or in multiple genes. Thus, the present disclosure provides for libraries of genotypically different strains of a microorganism that comprises a single gene mutation, edit or disruption, and also provides for combinatorial libraires of genotypically different strains of a microorganism that comprise two or more gene mutations or disruptions, the gene mutations, edits or disruptions can be in a single gene or more; in one implementation they are in different genes. In such a case, one may derive gene interaction terms (epistasis) for strain fitness, which may be beneficial for creating host microorganism strains that have improved strain fitness in tanks or bioreactors. In a particular implementation, the genotypically different strains of a microorganism comprise 1, 2, 3, 4, 5, 6, 7, 8, 9, 10, 11, 12, 13, 14, 15, 16, 17, 18, 19, 20, or a range that includes or is between any two of the foregoing values, of gene mutations, edits, or disruptions.

In regards to tags to identify each strain of the microorganism, the tag can be any molecule that allows for identification of the particular strain being studied or measured. Typically, such tags are nucleic acid-based tags, such as bar code sequences, that can be inserted into the strain genome by use of transposon or gene editing tools. Alternatively, the identifying tag may be a bioluminescent, fluorescent tags, marker genes, etc., that are knocked into the strain genome by use of gene editing or transposon-based systems.

In a further implementation, the disclosure provides a tank selection process to identify strain(s) of a microorganism that exhibit superior strain fitness in bioreactors and/or improved genotypes, comprising culturing genotypically different strains in one or more bioreactors, wherein, at various timepoints, samples are taken from the one or more bioreactors. The culture conditions for growing up the strains in the bioreactors can use batch processes, fed-batch process, continuous processes, or any combination of the foregoing. For example, the strain can be cultured first in one or more bioreactors using a batch process, and then switched to a fed-batch process once the strains have exhausted all the nutrients provided in the batch process, which can be indicated by a spike in dissolved oxygen. The microorganism used to make the genotypically different strains maybe be recombinantly modified to comprise a selectable marker(s), e.g., one or more genes for antibiotic resistance, or disruption of gene needed for growth (e.g., gene required to make an amino acid). Accordingly, the bioreactors may comprise antibiotic(s) and/or required nutrient(s) to select for the recombinantly modified organism or strains made therefrom. In a particular implementation, the microorganism comprises an antibiotic resistant gene as a selectable marker. Examples of antibiotic resistant genes include genes for resistance to: hygromycin, β-lactam antibiotics, aminocoumarin antibiotics, aminoglycoside antibiotics, chloramphenicol, ethambutol, mupirocin, peptide antibiotics, phenicol, rifampin, streptogramin antibiotics, fluoroquinolone antibiotics, fosfomycin, glycopeptide antibiotics, lincosamide antibiotics, linezolid, marcrolide antibiotics, tetracycline antibiotics, sulfonamide antibiotics, and streptothricin. The culture medium will comprise one or more nutrient sources (e.g., glucose) and buffering agents. The culture medium may further comprise: trace elements, vitamins, salts, succinate or fumarate, antibiotic(s), and/or anti-foaming agents. In one implementation, the samples that are taken from the one or more bioreactors at various time points are used to kinetically analyze the fitness of the genotypically different strains. In one implementation, certain strains are outcompeted by other strains over time and the rate at which certain strains are increasing while other strains are decreasing can be determined.

Accordingly, in a certain implementation, the disclosure provides a tank selection process to identify strain(s) of a microorganism that exhibit superior strain fitness in bioreactors and/or improved genotypes, comprising measuring the frequency of each genotypically different strain in the samples, wherein each genotypically different strain can be identified by the unique identifying tag. As noted above, the identifying tag can be a nucleic acid tag or a fluorescent or bioluminescent tag. In the case of the latter, the tag can be measured based upon light emissions and/or intensity. In the case of the former, the nucleic acid tag can be determined using sequencing, for example.

In another implementation, a tank selection process to identify strain(s) of a microorganism that exhibit superior strain fitness in bioreactors and/or improved genotypes, comprising assigning a quantitative fitness score for the genotypically different strains of the microorganism based upon how frequently the particular strain is present in the samples, wherein superior strain fitness in bioreactors is correlative with a high quantitative fitness score. The fitness score can be calculated using any suitable algorithms and software analysis. In one implementation, a fitness score higher than the mean indicates that the strain of that genotype is more fit in bioreactors than the average of the strains. A fitness score well above the mean indicates that the genotype of the strain has excellent strain fitness in bioreactors. Alternatively, a fitness score lower the mean indicates that the genotype of the strain is less fit in bioreactors than the average of the strains. A fitness score well below the mean indicates that the genotype of the strain has poor strain fitness in bioreactors. A fitness score, at or near the mean, is indicative that the genotype has limited impact on the strain's fitness in the bioreactors. Accordingly, the data generated from the fitness score of the genotypically different strains is invaluable as it provides clarity and certainty as to which mutations, edits, or disruptions are directly correlative to strain fitness in bioreactors. By using the fitness score data, one may model the microorganism genome for strain fitness and guide future library designs.

In a further implementation, the present disclosure provides that the samples from the tanks selection process can be singulated and assessed by a high throughput screening process to identify strains that have good tank fitness but also have improved genotypes. In a further implementation, the high throughput screening process comprises: (a) generating isolates from the plurality of genotypically different strains of the microorganism, wherein each isolate comprises only one strain of the microorganism, wherein each isolate can be identified by the unique identifying tag; and (b) assessing the isolates for improved genotypes in comparison to the microorganism or other isolates by using high throughput screening. In another implementation, the high throughput screening process is run in parallel with the tank selection process. In another implementation, the high throughput screening process is run overlaps or is run before or after the tank selection process. In one implementation, the improved genotype of the strain is in reference to product formation. However, an improved genotype may also be in reference to the strain's adaption to a particular environment, e.g., heat, alcohol or solvent concentration, etc. In regards to product formation, the product that is produced by the strain can be selected from the group consisting of a biofuel (e.g., methanol, ethanol, propanol, butanol, or isobutanol); a terpene or terpene containing compound (e.g., β-Carotene, camphor, menthol, limonene, linalool, geraniol, farnesene, squalene, capsidiol, and artemisinin); an alkaloid; a phenylpropanoid; a fatty acid-derived compound (e.g., free fatty acids, fatty alcohols, fatty acid ethyl esters, and fatty acid methyl esters); a polyketide; and a recombinant protein (e.g., an antibiotic, a restriction enzyme, a hormone, a protein therapeutic, a xylanase, a protease, a vaccine, a polyphenol oxidase, a laccase, and a cystatin).

In a further implementation, the disclosure provides that the processes of the disclosure (e.g., tank selection process or high throughput screening process), can also be used with cells (e.g., mammalian cells, insect cells, etc.). In such a case, the same gene editing and mutagenesis protocols and systems can be used to create genotypically different cells from a starting host cell. The host cell, like the microorganism, can be a wild type host cell, or recombinantly modified. The host cell can be isolated from a subject, or originate from a cell line. The host cell can be any type of cell including somatic cells, progenitor cells, stem cells, fibroblasts, cancer cells, chimeras, etc. The same tank selection process to generate fitness scores can be used with the genotypically different cells in bioreactors specialized for culturing cells, as opposed to microorganisms. The genotypically different cells can then be screened for improved genotypes using the high throughput selection methods described herein, but which have been modified for cell culture.

### Implementations

The following implementations are provided as illustrative of the disclosed methods and processes. These implementations are not intended to be limiting.

Implementation 1: A tank selection process for correlating a genotype from a strain of a microorganism with its fitness in a bioreactor(s), comprising:
(i) seeding one or more bioreactors with a plurality of genotypically different strains of a microorganism, wherein each strain of the microorganism comprises a unique identifying tag;
(ii) culturing the genotypically different strains in one or more bioreactors, wherein, at various time points, samples are taken from the one or more bioreactors;
(iii) measuring the frequency of each genotypically different strain in the samples, wherein each genotypically different strain can be identified by the unique identifying tag; and
(iv) assigning a quantitative fitness score for each of the genotypically different strains of the microorganism based upon how frequently the particular strain is present in the samples, wherein the value of the quantitative fitness score is correlative with the genotype from a strain and its fitness in bioreactor(s).

Implementation 2: The tank selection process of Implementation 1, wherein the microorganism is a bacterium or a yeast.

Implementation 3: The tank selection process of Implementation 2, wherein the yeast is selected from the group consisting of Saccharomyces cerevisiae, Pichia pastoris, Hansenula polymorpha, Kluyveromyces lactis, Schizosaccharomyces pombe, Yarrowia lipolytica, and Arxula adeninivorans.

Implementation 4: The tank selection process of Implementation 3, wherein the yeast is Saccharomyces cerevisiae.

Implementation 5: The tank selection process of Implementation 2, wherein the bacterium is selected from the group consisting of Escherichia Coli, Caulobacter crescentus, Rodhobacter sphaeroides, Pseudoalteromonas haloplanktis, Shewanella sp. strain Ac10, Pseudomonas fluorescens, Pseudomonas putida, Pseudomonas aeruginosa, Halomonas elongate, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, and Lactobacillus gasseri.

Implementation 6: The tank selection process of Implementation 5, wherein the bacterium is Escherichia Coli.

Implementation 7: The tank selection process of any one of the preceding Implementations, wherein the microorganism is a recombinantly modified microorganism.

Implementation 8: The tank selection process of Implementation 7, wherein the microorganism is recombinantly modified to express a biofuel, a terpene or terpene containing compound, an alkaloid, a phenylpropanoid, a fatty acid-derived compound, a polyketide, or a recombinant protein.

Implementation 9: The tank selection process of any one of the preceding Implementations, wherein the genotypically different strains of the microorganism are generated using mutagenesis or gene editing.

Implementation 10: The tank selection process of Implementation 9, wherein the genotypically different strains of the microorganism comprise a mutation, edit, or disruption in a single gene of the microorganism.

Implementation 11: The tank selection process of Implementation 9, wherein the genotypically different strains of the microorganism comprise 2 to 10 mutations, edits, or disruptions in one or more genes of the microorganism.

Implementation 12: The tank selection process of Implementation 9, wherein the genotypically different strains of the microorganism are generated by using a gene editing technology selected from the group consisting of CRISPR-Cas12a, CRISPR-Cas13, CRISPR-Cas9, TALEN, and Zinc Finger Nucleases.

Implementation 13: The tank selection process of Implementation 12, wherein the genotypically different strains of the microorganism are generated by using CRISPR-Cas12a.

Implementation 14: The tank selection process of Implementation 13, wherein the genotypically different strains of the microorganism are generated by using a digital genome engineering platform.

Implementation 15: The tank selection process of any one of the preceding Implementations, wherein the unique identifying tag is a barcode sequence.

Implementation 16: The tank selection process of Implementation 15, wherein the barcode sequence is incorporated into the genome of the different strains of the microorganism using CRISPR-Cas12a.

Implementation 17: The tank selection process of Implementation 16, wherein the CRISPR-Cas12a plasmids used to edit the strain's genome further comprise the barcode sequences.

Implementation 18: A screening process to identify strain(s) of a microorganism that exhibit superior strain fitness in bioreactors and improved genotypes, the screening process comprising a tank selection process and a high throughput screening process, the tank selection process comprising:
(i) seeding one or more bioreactors with a plurality of genotypically different strains of a microorganism, wherein each strain of the microorganism comprises a unique identifying tag;
(ii) culturing the genotypically different strains in one or more bioreactors, wherein, at various time points, samples are taken from the one or more bioreactors;
(iii) measuring the frequency of each genotypically different strain in the samples, wherein each genotypically different strain can be identified by the unique identifying tag;
(iv) assigning a quantitative fitness score for the genotypically different strains of the microorganism based upon how frequently the particular strain is present in the samples, wherein superior strain fitness in bioreactors is correlative with a high quantitative fitness score; and
the high throughput screening process comprising:
(a) generating isolates from the plurality of genotypically different strains of the microorganism, wherein each isolate comprises only one strain of the microorganism, wherein each isolate can be identified by the unique identifying tag; and
(b) assessing the isolates for improved genotypes in comparison to the microorganism or other isolates by using high throughput screening, wherein strain(s) of the microorganism that exhibit superior strain fitness in bioreactors and improved genotypes can be identified by having a high quantitative fitness score and exhibiting an improved genotype in high throughput screening.

Implementation 19: The screening process of Implementation 18, wherein the microorganism is a bacterium or a yeast.

Implementation 20: The screening process of Implementation 19, wherein the yeast is selected from the group consisting of Saccharomyces cerevisiae, Pichia pastoris, Hansenula polymorpha, Kluyveromyces lactis, Schizosaccharomyces pombe, Yarrowia lipolytica, and Arxula adeninivorans.

Implementation 21: The screening process of Implementation 20, wherein the yeast is Saccharomyces cerevisiae.

Implementation 22: The screening process of Implementation 21, wherein the bacterium is selected the group consisting of Escherichia Coli, Caulobacter crescentus, Rodhobacter sphaeroides, Pseudoalteromonas haloplanktis, Shewanella sp. strain Ac10, Pseudomonas fluorescens, Pseudomonas putida, Pseudomonas aeruginosa, Halomonas elongate, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, and Lactobacillus gasseri.

Implementation 23: The screening process of Implementation 22, wherein the bacterium is Escherichia Coli.

Implementation 24: The screening process of any one of Implementations 18 to 23, wherein the microorganism is a recombinantly modified microorganism.

Implementation 25: The screening process of Implementation 24, wherein the microorganism is recombinantly modified to express a biofuel, a terpene or terpene containing compound, an alkaloid, a phenylpropanoid, a fatty acid-derived compound, a polyketide, or a recombinant protein.

Implementation 26: The screening process of any one of Implementations 18 to 25, wherein the genotypically different strains of the microorganism are generated using mutagenesis or gene editing.

Implementation 27: The screening process of Implementation 26, wherein the genotypically different strains of the microorganism comprise a mutation, edit, or disruption in a single gene of the microorganism.

Implementation 28: The screening process of Implementation 26, wherein the genotypically different strains of the microorganism comprise 2 to 10 mutations, edits, or disruptions in one or more genes of the microorganism.

Implementation 29: The screening process of Implementation 26, wherein the genotypically different strains of the microorganism are generated by using a gene editing technology selected from the group consisting of CRISPR-Cas12a, CRISPR-Cas13, CRISPR-Cas9, TALEN, and Zinc Finger Nucleases.

Implementation 30: The screening process of Implementation 29, wherein the genotypically different strains of the microorganism are generated by using CRISPR-Cas12a.

Implementation 31: The screening process of Implementation 26, wherein the genotypically different strains of the microorganism are generated by using a digital genome engineering platform.

Implementation 32: The screening process of any one of Implementations 18 to 31, wherein the unique identifying tag is a barcode sequence.

Implementation 33: The screening process of Implementation 32, wherein the barcode sequence is incorporated into the genome of the different strains of the microorganism using CRISPR-Cas12a.

Implementation 34: The screening process of Implementation 33, wherein the CRISPR-Cas12a plasmids used to edit the strain's genome further comprise the barcode sequences.

Implementation 35: The screening process of any one of Implementations 18 to 34, wherein the high throughput screening process is run in parallel with the tank selection process.

Implementation 36: The screening process of any one of Implementations 18 to 35, wherein the high throughput screening process is used to determine and isolate strains which have an improved genotype for product formation.

Implementation 37: The screening process of Implementation 36, wherein the product that is formed is selected from the group consisting of a biofuel, a terpene or terpene containing compound, an alkaloid, a phenylpropanoid, a fatty acid-derived compound, a polyketide, and a recombinant protein.

Implementation 38: The screening process of Implementation 37, wherein the biofuel is an alcohol selected from the group consisting of methanol, ethanol, propanol, butanol, and isobutanol.

Implementation 39: The screening process of Implementation 37, wherein the terpene or terpene containing compound is selected from the group consisting of β-Carotene, camphor, menthol, limonene, linalool, geraniol, farnesene, squalene, capsidiol, and artemisinin.

Implementation 40: The screening process of Implementation 39, wherein the terpene or terpene containing compound is farnesene.

Implementation 41: The screening process of Implementation 37, wherein the fatty acid-derived compound is selected from the group consisting of free fatty acids, fatty alcohols, fatty acid ethyl esters, and fatty acid methyl esters.

Implementation 42: The screening process of Implementation 37, wherein the recombinant protein is selected from the group consisting of an antibiotic, a restriction enzyme, a hormone, a protein therapeutic, a xylanase, a protease, a vaccine, a polyphenol oxidase, a laccase, and a cystatin.

It should be appreciated that all combinations of the foregoing implementations and concepts are contemplated as being part of the inventive subject matter disclosed herein and may be employed in any combination to achieve the benefits described herein.

The following examples are intended to illustrate but not limit the disclosure. While they are typical of those that might be used, other procedures known to those skilled in the art may alternatively be used.

### Examples

Medium formulations
Outgrowth Media:

| | 1 | pH 5.0 | |
|---|---|---|---|
| Volume Created (L) | Vendor, | Expected | |
| Component | Cat# | Amount | unit |
| Deionized Water | N/A | 500 | mL |
| 400 g/L Glucose | From stock | 50 | mL |
| solution | | | |
| 5X Salts | From stock | 200 | mL |
| 0.5 M Succinate | From stock | 100 | mL |
| Sulfate solution | From stock | 4.65 | mL |
| 100X Trace Elements | From stock | 10 | mL |
| 83.33X vitamins | From stock | 12 | mL |

Production Media:

| | | pH: 5.0 | |
|---|---|---|---|
| Volume Created (L) | 1 | Expected | |
| Component | Vendor, Cat# | Amount | unit |
| De-ionized Water | N/A | 100 | mL |
| 100 g/L Galactose solution | From stock | 400 | mL |
| 400 g/L Glucose solution | From stock | 5 | mL |
| 5X Salts | From stock | 200 | mL |
| 0.5 M Succinate | From stock | 200 | mL |
| Sulfate solution | From stock | 4.65 | mL |
| 100X Trace Elements | From stock | 10 | mL |
| 83.33X vitamins | From stock | 12 | mL |

5X Salts:

| Volume created (L) | 1 | | Expected | |
|---|---|---|---|---|
| Component | Vendor | Cat # | Amount | Unit |
| DiWater | N/A | N/A | 750 | mL |
| Ammonium sulfate | Sigma | A5132 | 75 | g/L |
| Monopotassium | Sigma | 60218/P0662 | 40 | g/L |
| phosphate | | | | |
| Magnesium sulfate | Sigma | M1880 | 30.75 | g/L |
| heptahydrate | | | | |

83.33X Vitamins:

| | | | Expected | |
|---|---|---|---|---|
| Component | Vendor | Cat # | Amount | unit |
| D-Biotin | Sigma | 8512090001 | 50 | mg/L |
| DiWater | N/A | N/A | 750 | mL |
| Ca-D-pantothenate | Sigma | P5155 | 1 | g/L |
| Nicotinic Acid | Sigma | N0761 | 1 | g/L |
| Myo-inositol | Sigma | 17508 | 25 | g/L |
| Thiamine HCl | Sigma | T1270 | 1 | g/L |
| Pyridoxine HCl | Sigma | P6280 | 1 | g/L |
| p-aminobenzoic acid | Sigma | A9878 | 0.2 | g/L |

100X Trace Elements:

| | | | Expected | |
|---|---|---|---|---|
| Compound | Vendor | Cat# | Amount | unit |
| 0.5 M EDTA stock | From Stock | From Stock | 80.6 | mL/L |
| solution | | | | |
| Zinc Sulfate heptahydrate | Sigma | Z4750 | 5.75 | g/L |
| DiWater | N/A | N/A | 750 | mL |
| Iron Sulfate heptahydrate | Sigma | F7002 | 2.8 | g/L |
| Calcium Chloride | Sigma | C3881 | 2.9 | g/L |
| dihydrate | | | | |
| Copper Sulfate | Sigma | C8027 | 0.5 | g/L |
| pentahydrate | | | | |
| Sodium molybdate | Sigma | 480967 | 0.48 | g/L |
| dihydrate | | | | |
| Cobalt chloride | Sigma | C8661 | 0.47 | g/L |
| hexahydrate | | | | |
| Manganese chloride | Sigma | 203734 | 0.32 | g/L |
| tetrahydrate | | | | |

Seed Train. Seed 1: Seed vials were produced using the Onyx library output and stored at −80° C. with added glycerol. In order to maintain plasmid retention, hygromycin was added to each fermentation process. 30 uL of the Onyx library culture was inoculated into 50 mL of minimal medium with added hygromycin at final concentration of 0.375 mg/mL in a 250 mL baffled flask. The inoculum size was calculated to target initial cell density of 1×10⁶ cells in the first seed. After 24 hours of incubation the final OD was 5.9.

Seed 2: 0.5 mL of the culture from the first seed was then passaged into a second seed flask containing 50 mL minimal medium with hygromycin at 0.375 mg/mL in a 250 mL baffled flask with. A second shake flask was incubated for 24 hours and the final OD was 8.9. Cultivation conditions for both seed flasks were as follows: temperature was controlled at 30° C., agitation at 225 rpm in a 1-inch throw shaker to allow enough oxygenation. The seed medium was based upon the medium described in Westfall et al. (PNAS 109 (3): E111-E118 (2012)).

Main Fermentation in Bioreactors. The main batch fermentation medium was the same as described in Westfall et al. Three 250 mL bioreactors were used for the study. Each bioreactor prior to inoculation was batched with 90 mL of minimal medium, 150 uL of hygromycin stock at 250 mg/mL. 11 mL of isopropyl myristate was added as an organic overlay to allow product partitioning. The culture from the end of seed 2 was used as an inoculum for each bioreactor with target OD of 0.75 at inoculation time. 31 mL of seed 2 culture was diluted with 5.79 mL of seed medium to target OD of 7.5; each reactor was then inoculated with 10 mL of diluted culture to reach initial OD of 0.75. The temperature in the reactor was controlled at 30° C. and pH at 5.0 using 14% ammonium hydroxide. The dissolved oxygen was controlled at 30% by cascading agitation (from 600-2500 rpm) and then airflow (0.5-1 vvm). The initial glucose concentration in the batch phase was 19.5 g/L. The end of batch phase was evident by the second DO spike which coincided with exhaustion of batch glucose along with any produced ethanol.

After the batch phase, an exponential feed phase started. The starting feed rate was 0.01185 mL/min, with a volume basis of 100 mL initial volume. The growth rate of the feed was 0.2/h, and the exponential feed continued for 4.5 h or until OUR was >150 mmol/L/h. Once either of these triggers was reached, the exponential feed was ended and a constant low flow feed of 1 g glucose/L current volume/h was used.

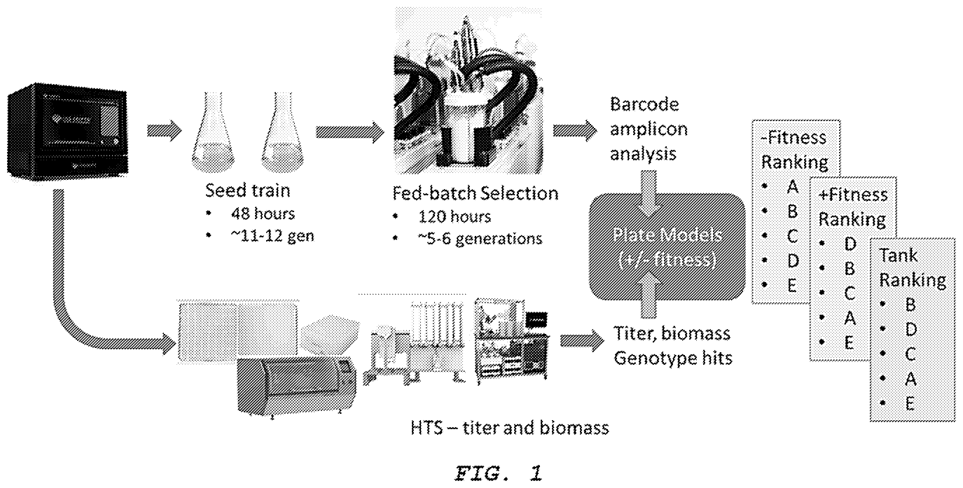

At the same time, a DO spike bolus feeding was turned on. The boluses feed 10 g glucose/L current volume over 30 minutes; they fed at a rate of 20 g glucose/L current volume/h during that time. Both feeds are based on current volumes in the bioreactors. The feed contained 585 g/L dextrose, 4.5 g/L potassium phosphate monobasic, 5.12 g/L magnesium sulfate heptahydrate, 35 mL sulfate solution, and 0.2 mg/mL hygromycin. Cell pellet samples were taken from each reactor every 12 hours and stored in YNBA media with 15% glycerol and stored in −80° C. freezer. These samples were later used for sequencing and also plated and singulated into colonies for rescreening in a high throughput screen. FIG. 1 provides a flow chart generically summarizing the process described in this Example.

### High Throughput Screening of Farnesene Produced by Clonal Isolates in a Growth Assay.

Outgrowth Plating (Colony Picking) (Day 1): Single colonies were obtained by plating to q-trays with hygromycin for selection. Colonies were picked into 96 well outgrowth plates containing 200 uL of outgrowth media using a Q-pix 420. Control strains including the parent strain 63 were included. The plates were sealed with AeraSeal™ film, and incubated at 30° C. with 1,000 rpm shaking (3 mm throw) and 80% humidity for 44 h (40 h-44 h).

Production Plating (dilution)—(Day 3): 5 uL of Outgrowth culture was transferred to a Production plate containing 195 uL of Production Media and 30 uL dodecane overlay. AeraSeal™ film were applied and plates were incubated for 48 h in an INFORS HT shaker at 30° C., 1,000 rpm (3 mm throw) and 80% humidity.

Production Plate Assaying—(Day 5): Production plates were extracted by addition of 110 uL of butanol to each well. Plates were sealed and incubated for one hour at 30° C., 1,000 rpm (3 mm throw). Plates were then centrifuged at 3,500×g for 5 minutes and sampled for OD and titer assays.

Assay Preparation: For titer assay, 40 uL of the butanol layer from the production well is sampled into 60 uL of butanol in a destination plate. Titer plates were shaken for 10 minutes at 3,000 rpm on a MixMate then centrifuged for 1 minute at 500×g. For OD₆₀₀ biomass assay, 5 uL of the aqueous layer/cell mixture was resuspended and sampled into 45 μL of DI water.

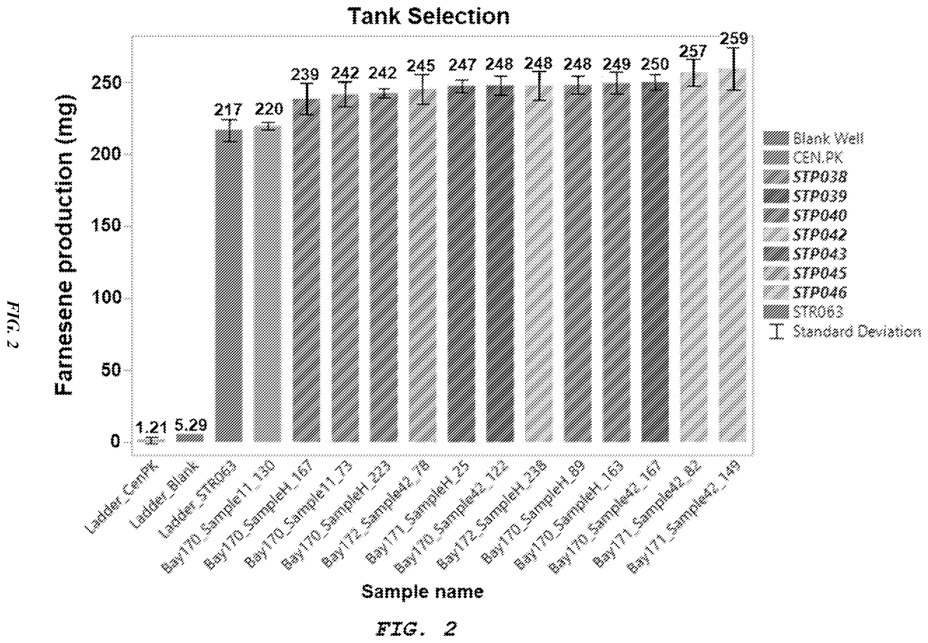

Farnesene Titer Assay: Farnesene titers were measured by reverse phase HPLC on an Agilent 1290 system. 1.5 uL of the extracted butanol layer was injected into a 2×30 mm C18 column with gradient of 10% ACN in water to 99% ACN in water (linear, 30 second method). Farnesene was detected at 220 nm with UV-vis detector, calibrated to external standard curve. FIG. 2 provides results. Error bars are standard deviation. Biomass assay: OD₆₀₀ measurements were made on a standard plate reader.

It will be understood that various modifications may be made without departing from the spirit and scope of this disclosure. Accordingly, other implementations are within the scope of the following claims.

What is claimed is:

1. A process, comprising:
(1) culturing a library of genetically modified strains of a microorganism in a bioreactor, wherein each strain in the library comprises a unique tag and a unique genetic modification; and
(2) determining fitness of a strain of the library from a sample from the bioreactor, using at least an amount of that strain present in the sample.

2. The process of claim 1, wherein determining fitness of the strain in the library comprises quantifying the amount of the strain that is present in the sample and/or measuring how frequently the strain is present in the sample.

3. The process of claim 1, wherein the microorganism is a bacterium or a yeast.

4. The process of claim 3, wherein the microorganism is a yeast selected from the group consisting of Saccharomyces cerevisiae, Pichia pastoris, Hansenula polymorpha, Kluyveromyces lactis, Schizosaccharomyces pombe, Yarrowia lipolytica, and Arxula adeninivorans.

5. (canceled)

6. The process of claim 3, wherein the microorganism is a bacterium selected from the group consisting of Escherichia coli, Caulobacter crescentus, Rodhobacter sphaeroides, Pseudoalteromonas haloplanktis, Shewanella sp. strain Ac10, Pseudomonas fluorescens, Pseudomonas putida, Pseudomonas aeruginosa, Halomonas elongate, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, and Lactobacillus gasseri.

7. (canceled)

8. The process of claim 1, wherein the unique genetic modification comprises (1) an insertion, deletion, or substitution of one or more nucleic acids; and/or (2) a deletion of an endogenous gene, downregulation of an endogenous gene, or upregulation of an endogenous gene.

9. (canceled)

10. The process of claim 1, wherein the unique genetic modification is made using a gene editing technology selected from the group consisting of CRISPR-Cas12a, CRISPR-Cas13, CRISPR-Cas9, TALEN, and Zinc Finger Nucleases.

11. The process of claim 1, wherein each strain in the library is recombinantly modified to (1) express a heterologous gene; and/or (2) produce or increase production of a biofuel, a terpene or terpene containing compound, an alkaloid, a phenylpropanoid, a fatty acid-derived compound, a polyketide, or a recombinant protein.

12. (canceled)

13. (canceled)

14. The process of claim 1, wherein the unique tag is a barcode sequence.

15. (canceled)

16. The process of claim 1, wherein the fitness is determined for each strain in the library.

17. The process claim 1, wherein determining fitness comprises assigning a quantitative fitness score to at least one strain in the library.

18. The process of claim 1, wherein a sample is removed from the bioreactor at two or more time points.

19. The process of claim 1, further comprising assessing one or more strains in the library by high throughput screening.

20. The process of claim 19, wherein the high throughput screening comprises determining production titer of a product.

21. The process of claim 20, wherein the product is selected from the group consisting of a biofuel, a terpene or terpene containing compound, an alkaloid, a phenylpropanoid, a fatty acid-derived compound, a polyketide, and a recombinant protein.

22. The screening process of claim 21, wherein the biofuel is an alcohol selected from the group consisting of methanol, ethanol, propanol, butanol, and isobutanol.

23. The screening process of claim 21, wherein the terpene or terpene containing compound is selected from the group consisting of β-Carotene, camphor, menthol, limonene, linalool, geraniol, farnesene, squalene, capsidiol, and artemisinin.

24. The screening process of claim 21, wherein the fatty acid-derived compound is selected from the group consisting of free fatty acids, fatty alcohols, fatty acid ethyl esters, and fatty acid methyl esters.

25. The screening process of claim 21, wherein the recombinant protein is selected from the group consisting of an antibiotic, a restriction enzyme, a hormone, a protein therapeutic, a xylanase, a protease, a vaccine, a polyphenol oxidase, a laccase, and a cystatin.

26.-40. (canceled)

41. The process of claim 1, further comprising seeding the bioreactor with the library of genetically modified strains prior to step (1).
